(19) United States
(12) Patent Application Publication
(10) Pub. No.: US 2021/0315810 A1
(43) Pub. Date: Oct. 14, 2021

(54) COMPOSITION CONTAINING A MUCOLYTIC AGENT FOR THE TREATMENT OF MUCUS HYPERSECRETION AND A DEVICE FOR THE DOSING THEREOF

(71) Applicant: SOFAR S.P.A., Trezzano Rosa [MI] (IT)

(72) Inventor: Andrea BIFFI, Trezzano Rosa [MI] (IT)

(21) Appl. No.: 17/265,195

(22) PCT Filed: Aug. 7, 2019
(86) PCT No.: PCT/IB2019/056710

§ 371 Date: Feb. 1, 2021

(30) Foreign Application Priority Data
Aug. 7, 2018 (IT) 102018000007928

(51) Int. Cl. A61K 9/00; A61K 47/36; A61K 47/26; A61K 31/198; A61K 47/12; A61M 15/00

(52) U.S. Cl. CPC A61K 9/0075; A61K 47/36; A61K 47/26; A61M 2210/1039; A61K 47/12; A61M 15/0065; A61M 2202/064; A61K 31/198

(57) ABSTRACT

The present invention regards a composition in powder form for oral inhalation, preferably for oral aspiration, comprising a mucolytic agent, preferably N-acetylcysteine. Furthermore, the present invention regards said composition for use in a method for the treatment of a mucus hypersecretion and of diseases, symptoms or disorders associated with it in a needy subject. Lastly, the present invention regards a device for administration, through inhalation route by means of oral aspiration, to said needy subject.

The present invention regards a composition in powder form (dry powder) for oral inhalation, preferably for oral aspiration, comprising a mucolytic agent, preferably N-acetylcysteine. Furthermore, the present invention regards said composition for use in a method for the treatment of a mucus hypersecretion (or bronchial mucus) and of diseases, symptoms or disorders associated with it in a needy subject. Lastly, the present invention regards a dispensing device for the administration, through inhalation route by means of oral aspiration, of said composition in form of dry powder to said needy subject.

The respiratory tracts and the lungs (respiratory system) are affected by serious diseases caused by bacteria, viruses and toxic substances that can easily penetrate into the respiratory system along with the inspired air. Inflammations, infections or allergies of the respiratory system are generally associated with a mucus hypersecretion. In physiological situations the mucus of the respiratory system (or bronchial mucus), a fluid and stringy substance, serves for moistening the respiratory tract and for trapping any possible foreign particles and microorganisms. In case of inflammations or infection or allergy of the respiratory system, the production of mucus increases starting off a mucus hypersecretion (or formation of phlegm) with increased consistency (viscosity). Said mucus hypersecretion has the purpose of capturing and eliminating, by coughing, the pathogen microorganisms that triggered the aforementioned inflammation or infection or allergy.

The alterations of production of mucus and the mucociliary clearance are generally treated using mucolytic/mucoregulatory drugs and with expectorant drugs. Mucolytic/mucoregulatory drugs act on mucus hypersecretion and on the characteristics of the mucus produced and they are used as symptomatic and adjuvant drugs when treating inflammatory diseases, infections or allergies regarding the respiratory tract. Mucolytic/mucoregulatory agents are capable of modifying the chemical/physical properties of the bronchial secretions facilitating the expectoration thereof and reducing the production thereof. Given that the hyperproduction of mucus and the modification of the characteristics thereof (e.g. Increased viscosity) can generate symptoms of the obstructive type, which force the system to utilise the cough reflex to clear the airways, the drugs active on the mucosal secretion take an active part in controlling the cough too.

There are two types of mucolytic/mucoregulatory drugs, depending on their action mechanism:
direct action, or fluidifying mucolytic/mucoregulatory drugs which are drugs capable of acting on the mucus already formed or present in the respiratory tracts, for example compounds that split the mucus polymers (e.g. N-acetylcysteine);
indirect action mucolytic/mucoregulatory drugs, capable of acting on the production of mucus by the mucus-secretory cells. These modify the secretion and characteristics of the mucus (e.g. sobrerol and carbocisteine) or the adhesiveness of the mucus (e.g. ambroxol and bromhexine).

Drugs and other remedies currently available in the market for the treatment of mucus hypersecretion and diseases, symptoms or disorders associated with them are often ineffective or only partially effective. Furthermore, some of the prior art products may cause adverse effects such as taste disorders, intestinal disorders, nausea, dyspepsia, vomit, dry mouth, abdominal pain, at times urticaria, skin rash, erythema, dermatitis, vertigo, dyspnoea, angioedema.

Furthermore, most of the drugs and other remedies currently available on the market for the treatment of mucus hypersecretion are in form of solutions or suspensions. When said solutions or suspensions are to be administered to the lungs through inhalation, this occurs using nebulisers. These devices dispense solutions and suspensions in form of thin spray and they generally have a face mask attached, so that the subject can inhale the thin spray through the mouth or nose. However, nebulisers tend to be large and generally non-portable devices, hence inappropriate for an easy and convenient administration of a medicinal product, like in the case of the present invention.

A further problem associated with the use of nebulisers lies in the difficulty of obtaining accurate information on the dose actually dispensed to the subject. There is also a general lack of precision, reproducibility and efficiency when it comes to administering the medicinal product, which leads to the need to increase the administered dose so as to guarantee the achievement of the desired therapeutic effect, with ensuing waste of the drug and higher risk of adverse effects.

When said drugs are in form of thin powder for inhalation administration, they must be administered using a dispensing device. The dispensing devices currently available on the market do not always guarantee an effective administration of the drug and they are often difficult to handle and activate.

In addition, it is known that delivery of compositions in form of dry powder to the respiratory tract reveals some drawbacks. The inhaler device should provide the maximum possible proportion of the active particles ejected into the lungs, including a significant proportion in the deepest part of the lungs. Thus, dry powder compositions capable of increasing the proportion of the active particles that are delivered to the lower respiratory tract or to the deep part of the lungs.

The type of dry powder inhaler used will impact the efficiency of release of the active particles to the respiratory tract. Furthermore, the chemical/physical properties of the powder affect both the efficiency and the reproducibility of the release of active particles and on the deposition site in the respiratory tract.

Thus, there is high demand from the society for appropriate treatments of mucus hypersecretion (or bronchial mucus) and of diseases, symptoms or disorders associated with them.

The technical problem addressed and solved by the present invention lies in providing a valid solution for the effective therapeutic and non-therapeutic treatment of mucus hypersecretions and of diseases, symptoms and/or disorders associated with said mucus hypersecretions capable of overcoming the currently unresolved drawbacks of the prior art, in particular, as regards the ineffectiveness and/or presence of adverse effects and/or administration challenges.

Furthermore, the technical problem addressed and solved by the present invention lies in providing new compositions, comprising—as active agent—a mucolytic agent in form of dry powder, suitable for inhalation administration having a good flowability, a good uniformity of distribution of the active agent, an appropriate chemical and physical stability prior to use and that do not tend to agglomerate, hence, that are easy to handle and administer.

In addition, the technical problem addressed and solved by the present invention lies in providing both new compositions in form of dry powder and a device for the inhalation administration thereof capable of creating a good breathable fraction and providing an accurate therapeutically active dose of the active ingredient.

In order to overcome these technical problems, the present invention provides a composition in form of powder, preferably dry powder, suitable for inhalation administration by means oral aspiration comprising a mucolytic agent (active agent), preferably N-acetylcysteine, and optionally other components (first and/or second support agent) which facilitate the administration of said composition in form of powder, preferably dry powder, through inhalation route by means of oral aspiration. Such composition is capable of effectively and quickly treating mucus hypersecretion and diseases, symptoms or disorders associated with said mucus hypersecretion, both in pathological subjects and healthy subjects (not yet defined pathological). In addition, said composition is without the adverse effects present in the treatments of the prior art, it is easy to administer through the inhalation route, it is easy to prepare and it is cost-effective.

Furthermore, the present invention provides a device for administering the composition according to the invention in form of powder for inhalation, preferably inhalation actuated through oral aspiration, that is easy to use, that allows to improve the adherence of the subject to the therapy, and having optimal capacity for administration of the composition of the invention and, thus, of the active ingredient comprised therein.

These and other objects, which will be clear from the detailed description that follows, are attained by the composition of the present invention due to the technical characteristics claimed in the attached claims.

Following an intensive research and development, the Applicant found out that the administration—through inhalation route by means of oral aspiration—of the composition according to the present invention is capable of effectively and quickly treating mucus hypersecretion and diseases, symptoms and/or disorders associated with said mucus hypersecretion.

Furthermore, following an intensive research and development, the Applicant found out that the use of a dispensing device of the invention for the administration—through oral inhalation, preferably by means of oral aspiration—of the composition of the invention in powder form improves the administration of the composition of the invention and, thus, of the active ingredient comprised therein.

Furthermore, said device is easy to use, hence, it improves the adherence of the subject to the therapy.

Said therapeutic or non-therapeutic pharmacological treatment activity of the composition is due to the mucolytic present in the composition, such as for example N-acetylcysteine, the additional components, if present in the composition besides the mucolytic, which allow to obtain an optimal powder for the administration thereof through the inhalation route and the dispensing device by means of which the composition of the invention in powder form is administered in the respiratory system through oral aspiration.

### FIGURES

FIG. 1: chart representing the average viscosity values of mucus collected from subjects suffering from cystic fibrosis before and after the treatment of the mucus using saline solution or using the Compositions 1-4 according to the invention (ETA: average viscosity value measured at 37° C. in mPa·s).

FIG. 2: chart representing the average viscosity values of mucus collected from subjects suffering from cystic fibrosis before and after the treatment of the mucus using saline solution or using the Compositions 5-12 according to the invention (ETA: average viscosity value measured at 37° C. in mPa·s).

FIG. 3: single-dose refillable device of the invention.

FIG. 4: single-dose refillable device of the invention, wherein the loader is in form of capsule.

### DETAILED DESCRIPTION OF THE INVENTION

Forming an object of the present invention is a composition in form of powder (dry powder) suitable for administration through oral inhalation (in short composition of the invention or composition) comprising (i) a mixture M (in short mixture of the invention or mixture) comprising, or alternatively, consisting of a mucolytic agent (active ingredient of the composition of the invention) and, optionally, (ii) at least one acceptable pharmaceutical or food grade additive and/or excipient.

In a preferred embodiment, said mucolytic agent is N-acetylcysteine or an acceptable pharmaceutical or food grade salt thereof, for example the L-lysine salt of N-acetylcysteine (NAL).

N-acetylcysteine (IUPAC name 2R-acetamido-3-sulfanylpropanoic acid, CAS 616-91-1) is a derivative of N-acetylate of the cysteine amino acid which has an antioxidant and mucolytic activity. Antioxidants are substances that slow or prevent the oxidation of other substances. Mucolytics are substances that make the mucus secreted by the respiratory system more fluid and facilitate the work of ejecting the mucus by the bronchi and trachea. It is known that the major determinants of viscosity and elasticity of the secretions of the respiratory system are fucomucins and IgG immunoglobulins. N-acetylcysteine, in particular, is characterised by the capacity to split the sulphur bridges in proteins: in the case of mucus, N-acetylcysteine depolymerises the mucoprotein complexes (glycoprotein agglomerates) into smaller units, provided with lower viscosity, and it exercises an important mucolytic and fluidifying effect on the mucosal and mucopurulent secretions effect.

In an embodiment, said (i) mixture M comprises, besides the mucolytic agent, preferably N-acetylcysteine or an acceptable pharmaceutical or food grade salt thereof (e.g. NAL), also a first support agent selected from among the group comprising or, alternatively, consisting of lactose, a dextran, mannitol and the mixtures thereof; more preferably lactose.

Preferably, said (i) mixture M comprises N-acetylcysteine and lactose.

Lactose (IUPAC name 3-D-galactopyranosyl (1→4) D-glucopyranose, CAS 63-42-3) is a disaccharide and a dextrorotatory reducing sugar. The lactose molecule consists of a D-galactose and of a D-glucose molecule linked by a glyosidic bond (acetalic) β(1-4). The aldehyde group of the glucose unit is responsible for the reducing properties of lactose. Lactose is added to the inhalation powders of the present invention to improve the efficiency at which the blister empties after respiratory activation, the turbulence and the dispersion of the drug in the small airways. The lactose particles have a particle diameter (>50 μm) such that they cannot penetrate into the deep parts of the respiratory system, hence, most of the lactose settles in the oropharynx before moving on to the stomach after being swallowed.

In an embodiment, said (i) mixture M comprises, besides the mucolytic agent, preferably N-acetylcysteine or a salt thereof, and, optionally, said first support agent selected from among the group comprising or, alternatively, consisting of lactose, a dextran, mannitol and the mixtures thereof, also a second support agent, wherein said second agent is a stearate metal or a derivative thereof selected from among the group comprising or, alternatively, consisting of: magnesium stearate, zinc stearate, calcium stearate, sodium stearate, lithium stearate, sodium stearyl fumarate, sodium stearoyl lactylate and the mixtures thereof; more preferably magnesium stearate.

Thus, the (i) mixture M may comprise said second support agent as an alternative to said first support agent or in presence of said first support agent.

In an embodiment, said (i) mixture M comprises, besides the mucolytic agent, preferably N-acetylcysteine or an acceptable pharmaceutical or food grade salt thereof, and lactose, also magnesium stearate.

Preferably, said (i) mixture M comprises N-acetylcysteine, lactose and magnesium stearate.

Alternatively, said (i) mixture M comprises N-acetylcysteine and magnesium stearate, in absence of lactose.

Magnesium stearate (IUPAC name magnesium octadecanoate, CAS 557-04-0) is used in the pharmaceutical industry as a lubricant in the preparation solid of compositions, preferably tablets, for facilitating the detachment between the powder or granulate and the metallic walls of the processing equipment.

In a preferred embodiment, said (i) mixture M comprises, besides the mucolytic agent, preferably N-acetylcysteine or a salt thereof, also a hyaluronic acid or an acceptable pharmaceutical or food grade salt thereof.

In an embodiment, said (i) mixture M comprises said mucolytic agent, preferably N-acetylcysteine or a salt thereof, hyaluronic acid or a salt thereof and said first support agent, preferably lactose.

In an embodiment, said (i) mixture M comprises said mucolytic agent, preferably N-acetylcysteine or a salt thereof, hyaluronic acid or a salt thereof and said second support agent, preferably magnesium stearate.

In an embodiment, said (i) mixture M comprises said mucolytic agent, preferably N-acetylcysteine or a salt thereof, hyaluronic acid or a salt thereof and said first support agent, preferably lactose, and said second support agent, preferably magnesium stearate.

In a preferred embodiment, said (i) mixture M comprises, besides the mucolytic agent, preferably N-acetylcysteine or an acceptable pharmaceutical or food grade salt thereof, lactose and/or magnesium stearate, also a hyaluronic acid or an acceptable pharmaceutical or food grade salt thereof.

The presence of hyaluronic acid in the composition of the invention combined with said mucolytic agent, preferably N-acetylcysteine, boosts the mucolytic effectiveness of the mucolytic agent (reduction of mucus viscosity and ease of expectoration/elimination of the mucus for the subject suffering from mucus hyperproduction). Thus, in the compositions of the invention it is possible to use a low % of mucolytic agent, preferably N-acetylcysteine.

In the context of the present invention, the expression hyaluronic acid salt is preferably used to indicate a salt of an alkaline metal or of an alkaline earth metal, such as for example sodium, potassium, magnesium or calcium; preferably the hyaluronic acid salt is the sodium salt (sodium hyaluronate).

Hyaluronic acid (CAS 9004-61-9) is a non-sulfated glycosaminoglycan and without protein core. Hyaluronic acid and the salts thereof are macromolecules. In particular, hyaluronic acid or the salt thereof, preferably sodium hyaluronate, in the context of the present invention preferably has an average molecular weight comprised between 20 kDa and 4000 kDa, preferably comprised between 50 kDa and 1500 kDa, even more preferably comprised between 150 kDa and 1000 kDa.

In an embodiment, the composition of the invention comprises said (i) mixture M comprising, or alternatively, consisting of: a concentration (%) by weight of the mucolytic agent, preferably N-acetylcysteine or a salt thereof, comprised in the range between 50% and 95% with respect to the total weight of the mixture M, preferably between 55% and 90%, more preferably between 60% and 85%; and, if present, a concentration (%) by weight of lactose, comprised in the range between 5% (or 4.9% if present magnesium stearate) and 40% with respect to the total weight of the mixture M, preferably between 10% and 35%, more preferably between 15% and 30%; and, if present, a concentration (%) by weight of magnesium stearate, comprised in the range between 0.1% and 20% with respect to the total weight of the mixture M, preferably between 0.5% and 15%, more preferably between 1% and 10%.

In an alternative embodiment, the composition of the invention comprises said (i) mixture M comprising, or alternatively, consisting of: a % by weight of N-acetylcysteine (present as such or in form of salt) comprised in the range between 0.1% and 60% with respect to the total weight of the mixture M, preferably between 5% and 50%, more preferably between 10% and 40%.

In an alternative embodiment, the composition of the invention comprises said (i) mixture M comprising, or alternatively, consisting of: a % by weight of N-acetylcysteine (present as such or in form of salt) comprised in the range between 0.1% and 60% with respect to the total weight of the mixture M, preferably between 5% and 50%, more preferably between 10% and 40%; and a % by weight of hyaluronic acid (present as such or in form of salt) comprised in the range between 40% and 90% with respect to the total weight of the mixture M, preferably between 50% and 80%, more preferably between 60% and 80%.

In a further preferred embodiment, the composition of the invention comprises said (i) mixture M comprising, or alternatively, consisting of: a % by weight of N-acetylcysteine (present as such or in form of salt) comprised in the range between 0.1% and 58% with respect to the total weight of the mixture M, preferably between 5% and 50%, more preferably between 10% and 40%; and a % by weight of hyaluronic acid (present as such or in form of salt) comprised in the range between 40% and 90% with respect to the total weight of the mixture M, preferably between 50% and 80%, more preferably between 60% and 80%; and, if present, a % by weight of a first support agent, preferably lactose, or of a second support agent, preferably magnesium stearate, comprised in the range between 2% and 35% with respect to the total weight of the mixture M, preferably between 10% and 30%, more preferably between 15% and 25%.

In a further preferred embodiment, the composition of the invention comprises said (i) mixture M comprising, or alternatively, consisting of: a % by weight of N-acetylcysteine (present as such or in form of salt) comprised in the range between 0.1% and 50% with respect to the total weight of the mixture M, preferably between 5% and 50%, more preferably between 10% and 40%; and a % by weight of hyaluronic acid (present as such or in form of salt) comprised in the range between 40% and 90% with respect to the total weight of the mixture M, preferably between 50% and 80%, more preferably between 60% and 80%; and, if present, a % by weight of a first support agent, preferably lactose, and of a second support agent, preferably magnesium stearate, comprised in the range between 10% and 40% with respect to the total weight of the mixture M, preferably between 15% and 35%, more preferably between 20% and 30%.

Examples of % by weight of the mixture M of the invention are indicated in Table 1 and Table 4.

The composition of the invention is in form of powder, preferably a dry powder having said composition in form of powder, preferably dry powder, a particle size diameter (average particle diameter) such to make the composition suitable to be administered through oral inhalation route. Preferably, the composition of the invention in powder form (dry powder) has a particle size diameter (average particle diameter) comprised in the range between 1 μm and 200 μm, preferably between 1 μm and 100 μm, more preferably between 1 μm and 50 μm.

In a preferred embodiment, the composition of the invention in powder form comprising NAC and, optionally, said first support agent and second support agent and/or hyaluronic acid, or the salts thereof, the average particle diameter comprised in the range between 0.1 μm and 10 μm, preferably between 0.5 μm and 5 μm, more preferably between 1 μm and 5 μm.

In the context of the present invention, the expression “average particle diameter” refers to the size of a particle comprising all components of the composition, for example, depending on the embodiment, of a particle comprising NAC or NAC and HA or NAC and HA and lactose or NAC and HA and magnesium stearate.

The powder particles may mainly be classified into inhalable and breathable. The inhalable fraction is represented by a suspension of particles of various diameters (generally comprised between 10 microns and 100 microns) whose dimensions are such to determine the interaction with the human respiratory system.

The inhalable fraction is represented by a suspension of particles with grain size class (generally <4 microns) such to reach, due to the breathing motions, the non-ciliated part of the lung (alveolar area).

In the context of the present invention, the expression “dry powder” is used to indicate a powder having a low moisture content (water), for example a powder having a content comprised in the range between 0.01% and 10% by weight with respect to the total weight of the powder, preferably between 0.1% and 5%, more preferably between 0.1% and 3%.

The composition of the present invention, optionally in addition to said first support agent, preferably lactose, and, if present, to said second support agent, preferably magnesium stearate, and/or hyaluronic acid or a salt thereof, further (ii) pharmaceutical or food grade additives and/or excipients, i.e. a substance without therapeutic activity suitable for pharmaceutical or food use. In the context of the present invention the acceptable ingredients for pharmaceutical or food use comprise all auxiliary substances known to the man skilled in the art such as, by way of non-limiting example, diluents, solvents (including water, glycerine, ethyl alcohol), solubilisers, thickeners, sweeteners, flavourings, dyes, lubricants, surfactants, antimicrobials, antioxidants, preservatives, pH stabilisation buffers and the mixtures thereof.

The composition according to the present invention, besides the components of the (i) mixture M mentioned above (such as a mucolytic agent (NAC or a salt thereof) and, optionally, a first and a second support agent and/or a hyaluronic acid or a salt thereof) and, optionally, further (ii) additives and/or excipients, may also comprise further active ingredients such as, by way or non-limiting example, anti-inflammatories, antioxidants, antibacterial agents, disinfectants, vasoconstrictors, probiotics, extracts of medicinal plants, vitamins, mineral salts, further mucolytics and generally products for the treatment of inflammations or infections or allergies of the respiratory system.

In a preferred embodiment, the composition of the invention is prepared using the method for the mechanical mixing of the single components.

In an alternative embodiment, the composition of the invention is prepared using the spray-drying method. The composition of the invention may be a pharmaceutical composition, nutraceutical composition, dietary supplement product or food for special medical purposes or a medical device composition.

The expression “medical device” in the context of the present invention is used according to the meaning laid down by the Italian Legislative Decree no. 46, dated 24 Feb. 1997 or according to the new Medical Devices Regulation (UE) 2017/745 (MDR), i.e. it indicates a substance or another product, used alone or in combination, designated by the manufacturer to be used in humans for the diagnosis, prevention, control, therapy or attenuation of a disease, the product not exercising the main action, in or on the human body, for which it is designated, neither using pharmacological or immunology means nor by means of a metabolic process but the function thereof can be coadjuvated by such means.

Forming an object of the present invention is the composition of the invention as defined above (such as, comprising a mucolytic agent, preferably N-acetylcysteine or a salt thereof, and, optionally, a first and/or second support agent, such as lactose and magnesium stearate, and/or hyaluronic acid or a salt thereof) for use as medicament.

Furthermore, forming an object of the present invention is the composition of the invention as defined above (such as, comprising a mucolytic agent, preferably N-acetylcysteine or a salt thereof, and, optionally, a first and/or second support agent, such as lactose and magnesium stearate, and/or hyaluronic acid or a salt thereof) for use in a method for the preventive and/or curative treatment of a mucus hypersecretion and of a disease, symptom and/or disorder associated with said mucus hypersecretion, in a needy subject, preferably wherein said composition for use is administered through oral inhalation activated by an aspiration action by the subject.

In an embodiment, the composition of the invention is for use in a method for the treatment of inflammations or infections or allergies of the respiratory system associated with a mucus hypersecretion. Preferably, said inflammations or infections or allergies of the respiratory system are selected from among the group comprising or, alternatively, consisting of: asthma, chronic obstructive pulmonary disease (COPD), bronchitis, emphysema, cystic fibrosis, pertussis, pneumonia, pleuritis, bronchiolitis, cold, sinusitis, rhinitis, tracheitis, pharyngitis, laryngitis, epiglottitis and bronchiectasis.

Furthermore, forming an object of the present invention is a dispensing device (in short device of the invention or, simply, device) for the administration—through oral inhalation route—of the composition of the invention in powder form, preferably dry powder, to a needy subject, wherein said device releases an effective dose (predetermined effective dose) of the composition of the invention when actuated by the subject through oral aspiration (in short device of the invention or dispensing device of the invention).

The dispensing device of the invention is a state-of-the-art inhaler, without propellant, which releases an effective dose of the composition of the invention when actuated by the subject through oral aspiration.

In the context of the present invention, the expression “oral aspiration” or “oral aspiration action” is used to indicate that the subject inhales or exhales using the mouth in a single action (one-time aspiration action) at a force intensity such to allow the device of the invention to dispense a predetermined dose of the composition of the invention in powder form in the dispensing device in question.

Said predetermined dose of the composition of the invention that can be dispensed by the device of the invention by means of the oral aspiration action by the subject may vary according to the variation of the age of the subject, the physical size of the subject (number of Kg) and the type of disease or disorder being treated.

Preferably, the device of the invention allows, by means of only one oral aspiration action, the dispensing of a dose of composition of the invention in dry powder form comprised in the range between 10 mg and 70 mg, preferably between 20 mg and 50 mg, more preferably between 35 mg and 40 mg.

Alternatively, said dose of composition of the invention in dry powder form comprised in the range between 10 mg and 70 mg, preferably between 20 mg a 50 mg, more preferably between 35 mg and 40 mg, may be dispensed by the device of the invention by means of an n number of oral aspirations, wherein n is a number variable between 2 and 4, preferably 2.

Furthermore, with the aim of reaching the daily intake of the composition of the invention required for said subject, the administration of said predetermined dose, dispensed by the device of the invention through one or more oral aspiration actions of the subject, can be carried out only 1 time per day or repeated 2 or 3 or 4 or 5 or 6 times per day.

The dispensing device of the invention can be of the disposable or refillable type. In a preferred embodiment, the device of the invention is refillable with the possibility of having single-dose or multi-dose.

The dispensing device of the invention, when refillable, can be of the single-dose or multi-dose type; when the dispensing device is of the multi-dose type it may contain a dosing indicator. In a preferred embodiment, the dispensing device of the invention is of the disposable single-dose type.

The dispensing device of the invention for the administration of a dry powder through the oral-inhalation route, wherein said device comprises:
a compartment for containing the powder comprising the composition in form of dry powder according to any one of the embodiments of the invention;
an aspiration channel, wherein a first end is directly or indirectly connected to the powder containment compartment and a second end allows the subject to carry out the oral aspiration action; and, optionally,
a compartment for containing the loader, wherein said loader, single-dose or multi-dose, contains the composition of the invention in powder form;
and wherein said dispensing device of the invention is structured so as to dispense a dose of said composition following an oral aspiration action by the subject in need of said administration.

When the device of the invention is refillable, the composition of the invention is loaded into the powder containment compartment using said loader comprising the composition of the invention; said loader can be a single-dose loader (for example a capsule or a sachet) or a multi-dose loader, preferably a single-dose loader. Furthermore, the loader may be inside the device of the invention (internal loader) or outside the device of the invention (external loader).

In an embodiment of the refillable device, said loader is an external loader not housed in the device and the device is structured so as to allow the direct transfer of a single-dose of said composition from the external loader (single-dose or multi-dose) to the powder containment compartment (direct loading refillable device). For example, said single-dose external loader is a sachet containing the composition of the invention in dry powder form.

In an alternative embodiment of the refillable device, said loader is an internal loader housed in the device in a loader containment compartment and the device is structured so as to allow the indirect transfer of a single-dose of said composition from the internal loader (single-dose or multi-dose) to the powder containment compartment by actuating a pressure on the device which causes the opening of the internal loader (indirect loading refillable device) or, alternatively, by actuating a pressure on the internal loader. For example, said single-dose internal loader is a capsule containing the composition of the invention in dry powder form, for example, a capsule made of flexible material, such as an aluminium capsule.

The expression “single-dose” “refillable” dispensing device is used in the context of the present invention to indicate that a single-dose of the composition of the invention is contained in a single-dose container separated or separable from the dispensing device (for example a sachet or a capsule). Said single-dose of the composition of the invention is transferred from the single-dose container in the dispensing device in the powder containment compartment and, lastly, said single-dose of the composition of the invention is dispensed by the dispensing device to the subject through oral aspiration by the subject (for example by means of one or more aspiration actions). The advantage of the “single-dose” dispensing device lies in the fact that the subject does not risk taking more than one dose, thus guaranteeing maximum safety.

The expression “multi-dose” “refillable” dispensing device is used in the context of the present invention to indicate that a multi dose of the composition of the invention in powder form is contained in a multi-dose container separated or separable from the dispensing device. Such multi-dose container, containing a defined number of doses of the composition of the invention, is inserted into the dispensing device of the invention (in the loader containment compartment) before the dispensing of said defined number of doses. Subsequently, a single-dose of the composition of the invention is transferred from said multi-dose container into the dispensing device in the powder containment compartment, for example by means of pressure on the device or on the multi-dose container inserted into the device by a subject (for example pressure exerted by the hand of the subject on a part of the device or on the multi-dose container to load the single-dose of composition to be dispensed into the special powder containment compartment; indirect multi-dose refillable dispensing device). Lastly, the single-dose of the composition of the invention loaded into the dispensing device (in the loader containment compartment) is dispensed by the dispensing device to the subject through oral aspiration by the subject (for example by means of one or more aspiration actions).

The dispensing device of the invention, when of the single-dose refillable type, can be of the “direct loading” type (with external loader) or of the “indirect loading type” (with internal loader).

In an embodiment, the dispensing device of the invention is of the indirect loading single-dose refillable type in that the powder (dry powder) is contained in the device (internal container) and it is released into the dispensing device (in the powder containment compartment) upon pressing by the subject.

In an alternative embodiment, the dispensing device of the invention is of the direct loading single-dose refillable type.

The expression “direct loading” “single-dose” “refillable” device is used in the context of the present invention to indicate that the single-dose of the composition of the invention is contained in a single-dose container separated from the device (external container), for example a sachet. The single-dose of the composition of the invention is directly transferred from the single-dose container to the dispensing device of the invention (in the powder containment compartment) before dispensing, for example by means of the opening of the single-dose container (e.g. a sachet) by a subject and direct transfer of the composition of the invention in powder form from the single-dose container to the dispensing device, more precisely to the powder containment compartment. Lastly, the composition of the invention is dispensed by the dispensing device to the subject through oral aspiration by the subject (for example by means of one or more aspiration actions).

The expression “indirect loading” “single-dose” “refillable” device is used in the context of the present invention to indicate that the single-dose of the composition of the invention is contained in a single-dose container separated from the device, for example a capsule. Such container containing the composition of the invention is inserted into the dispensing device (in the loader containment compartment) before dispensing. An opening is subsequently created in said single-dose container, for example by exerting pressure on the dispensing device by a subject, so that the composition is released into the dispensing device, more precisely into the powder containment compartment. Lastly, the single-dose of the composition of the invention is dispensed by the dispensing device to the subject through oral aspiration by the subject (for example by means of one or more aspiration actions).

The dispensing device of the invention allows the dispensing of the composition of the invention in form of a powder cloud which is durable over time and guarantees a more effective inhalation as well as a uniform and optimal distribution of the active ingredient or active ingredients comprised in the composition in the respiratory system, leading to benefits in terms of therapeutic results.

Forming an object of the present invention is a method for the treatment of mucus hypersecretion and a disorder associated with said mucus hypersecretion, such as an inflammation or infection or allergy of the respiratory system, in a needy subject, wherein said treatment method provides for the administration of the composition of the invention as defined above through the inhalation route by means of oral aspiration by the subject using the dispensing device of the present invention.

Forming an object of the present invention is the non-therapeutic use of the composition of the invention as defined above for the non-therapeutic treatment of a mucus hypersecretion and of a disorder associated with said mucus hypersecretion in a needy subject, wherein said non-therapeutic use provides for the administration of said composition through the inhalation route by means of oral aspiration using the dispensing device of the present invention.

In a preferred embodiment, the non-therapeutic use of the composition according to the invention is for the non-therapeutic treatment of a disorder associated with said mucus hypersecretion such as an inflammation or infection or allergy of the respiratory system.

Preferably, said inflammation or infection or allergy of the respiratory system is selected from among the group comprising or, alternatively, consisting of: asthma, chronic obstructive pulmonary disease (COPD), bronchitis, emphysema, cystic fibrosis, pertussis, pneumonia, pleuritis, bronchiolitis, cold, sinusitis, rhinitis, tracheitis, pharyngitis, laryngitis, epiglottitis and bronchiectasis.

Furthermore, forming an object of the present invention is the use of said dispensing device (in short device of the invention of, simply, device) for the administration—through oral inhalation route—of the composition of the invention in powder form, preferably dry powder, to a needy subject, wherein said device releases an effective dose (predetermined effective dose) of the composition of the invention when actuated by the subject through oral aspiration (in short device of the invention or dispensing device of the invention).

Lastly, forming an object of the present invention is a kit comprising (a) the composition of the invention, according to any one of the embodiments of the invention, (b) the dispensing device according to the invention for administering the composition of the invention to a needy subject; (c) instructions for administering the composition of the invention by means of the (b) dispensing device; and, optionally, (d) an additional active ingredient, that can be in an intake form equal to and/or different from the composition of the invention.

The expression “treatment method” in the context of the present invention is used to indicate an action, comprising the administration of a substance, or mixture of substances or combination thereof, with the aim of eliminating, reducing/decreasing or preventing a pathology or disease and its symptoms or disorders.

Unless specified otherwise, the indication that a composition “comprises” one or more components or substances means that other components or substances can be present besides the one, or the ones, indicated specifically.

As illustrated in the experimental part outlined below, the compositions of the present invention comprising N-acetylcysteine, alone or combined with hyaluronic acid and/or lactose and/or magnesium stearate, in powder form, preferably dry powder, for inhalation by means of oral aspiration, are effective for the therapeutic and non-therapeutic treatment of mucus hypersecretion (or bronchial mucus) and of the various diseases, symptoms or disorders of the respiratory system associated with said bronchial mucus hypersecretion.

In addition, the administration of the compositions of the invention by means of the dispensing device of the invention actuated by the aspiration of the subject undergoing the administration is such to activate the administration of the effective dose and the maximum inherent effectiveness of the compositions of the invention.

The composition of the invention, according to any one of the embodiments defined in the present invention, shows a good flowability, a good uniformity of distribution of the active ingredient (mucolytic agent, N-acetylcysteine) and an appropriate chemical and physical stability prior to use.

Furthermore, the composition of the invention, when inhaled using the inhalation device of the invention actuated by means of a single aspiration action by a subject to whom the composition is administered, creates a good inhalable fraction and an accurate therapeutically active dose of the active ingredient (mucolytic agent, N-acetylcysteine or the salts thereof).

The expression “good flowability” refers to a composition that can be easily handled during the preparation method and it is capable of guaranteeing an accurate and reproducible administration of the therapeutically effective dose when administered using the device of the invention by means of the aspiration action of the subject. The flow characteristics can be evaluated by measuring the Carr index; a Carr index lower than 25 is usually used to indicate good flow characteristics.

The expression “distribution uniformity” refers to a composition in which, when mixing, the uniformity of the content of the active ingredient, expressed as the relative standard deviation (RSD), is lower than 5%.

The expression “chemically stable” refers to a formulation that meets the ICH Q1A guidelines referring to “Stability testing of new active substances (and medicinal products)”.

The expression “physically stable” refers to a formulation in which if several components of the dry powder particles of the composition of the invention are present, these components substantially do not separate during the method for preparing the dry powder and/or over the time comprised between the preparation and the use of the composition.

The tendency to separate can be evaluated according to Staniforth et al., J. Pharm. Pharmacol., 34.700-706, 1982, which is wholly incorporated herein for reference, and it is considered acceptable if the distribution of the active ingredient in the dry powder composition after the test, expressed as the relative standard deviation (RSD), does not change significantly with respect to that of the composition prior to the test.

The expression “inhalable fraction” refers to an index of the particle percentage of active ingredient (mucolytic agent, N-acetylcysteine or the salts thereof) which reach the lungs (deep area) in a subject. The inhalable fraction, also referred to as the fine particle fraction (FPF), is evaluated using appropriate in vitro apparatus such as Multistage Cascade Impactor or Multi Stage Liquid Impinger (MLSI) according to the procedures indicated in common pharmacopeia. FPF is calculated from the ratio between the dispensed dose and the fine particle mass (of fine particle dose, in short FPD). An inhalable fraction greater than 30% is an index of good inhalation performance.

The expression “accurate therapeutically active dose of the active ingredient” refers to a composition in which the variation between the average dispensed daily intake and the average emitted dose is equal to or lower than 15%, preferably lower than 10%.

In the context of the present invention, the expressions “active agent” and “active ingredient” are synonyms and they refer to the mucolytic agent, preferably N-acetylcysteine and the acceptable pharmaceutical or food grade.

Embodiments (Frnr) of the present invention are indicated below:

FR1. A composition in a dry powder form for oral inhalation, preferably for oral aspiration, comprising:

(i) a mixture M comprising, or alternatively, consisting of a mucolytic agent and, optionally,

(ii) at least one acceptable pharmaceutical or food grade additive and/or excipient.

FR2. A composition according to FR1, wherein said mucolytic agent is N-acetylcysteine or an acceptable pharmaceutical or food grade salt thereof.

FR3. A composition according to FR1 or FR2, wherein said (i) mixture M further comprises lactose.

FR4. A composition according to any one of FR1 to FR3, wherein said (i) mixture M further comprises magnesium stearate.

FR5. A composition according to any one of FR1 to FR4, wherein said (i) mixture M further comprises a hyaluronic acid or an acceptable pharmaceutical or food grade salt thereof.

FR6. The composition according to any one of FR1 to FR5 wherein the (i) mixture M comprises or, alternatively, consists of: a concentration by weight of the mucolytic agent, preferably N-acetylcysteine or a salt thereof, comprised in the range between 50% and 95% with respect to the total weight of the mixture M, preferably between 55% and 90%, more preferably between 60% and 85%; of a concentration by weight of lactose, comprised in the range between 5% and 40% with respect to the total weight of the mixture, preferably between 10% and 35%, more preferably between 15% and 30%; and, if present, a concentration by weight of magnesium stearate, comprised in the range between 0.1% and 20% with respect to the total weight of the mixture, preferably between 0.5% and 15%, more preferably between 1% and 10%.

FR7. The composition according to any one of FR1 to FR6, wherein said composition is for use in a method for the preventive and/or curative treatment of a mucus hypersecretion and of a disease, symptom and/or disorder associated with said mucus hypersecretion, in a needy subject.

FR8. The composition for use according to FR7, wherein said disease, symptom and/or disorder associated with said mucus hypersecretion is an inflammation or infection or allergy of the respiratory system, preferably wherein said inflammation or infection or allergy of the respiratory system is selected from among the group comprising or, alternatively, consisting of: asthma, chronic obstructive pulmonary disease (COPD), bronchitis, emphysema, cystic fibrosis, pertussis, pneumonia, pleuritis, bronchiolitis, cold, sinusitis, rhinitis, tracheitis, pharyngitis, laryngitis, epiglottitis and bronchiectasis.

FR9. Dispensing device for the administration—through oral inhalation route—of the composition in form of powder according to any one of FR1 to FR6 to said needy subject, wherein said device releases an effective dose of said composition when actuated by the subject by means of oral aspiration.

FR10. Kit comprising:

(a) the composition according to any one of FR1 to FR6, preferably wherein said composition is contained in a container;

(B) the device according to FR9; and

(c) instructions on the method of use of said (a) composition and of said (b) device.

FR11. Non-therapeutic use of the composition according to any one of FR1 to FR6, wherein said use is for the non-therapeutic treatment of a mucus hypersecretion and of a disorder associated with said mucus hypersecretion in a needy subject, wherein said non-therapeutic use provides for the administration of said composition through the inhalation route by means of oral aspiration using the dispensing device according to claim 9.

### EXPERIMENTAL PART

Experimental Part (I)

The viscosity of the mucus coming from patients suffering from cystic fibrosis, whose mucus has particularly altered viscoelastic characteristics, was measured with the aim of the in vitro evaluation of the compositions according to the present invention (Composition 1-4 having the compositions indicated in table 1). Only samples of mucus with a viscosity comprised between 4.000 mPa·s. and 10.000 mPa·s. (millipascal×second or PI: Poiseuille) measured at the temperature of 37° C., were considered for the purposes of the present evaluation.

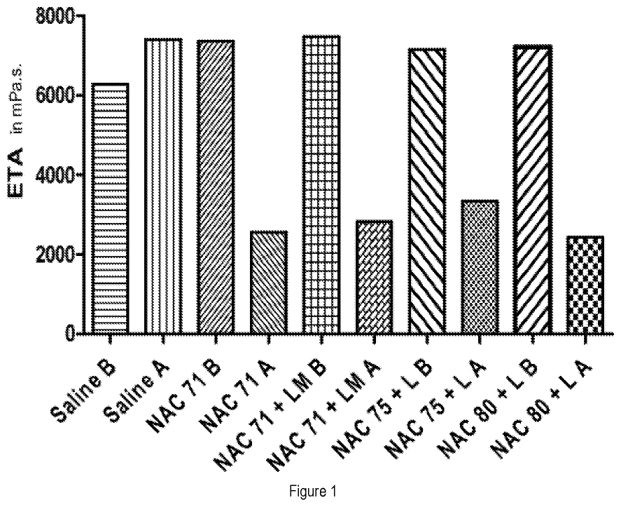

The viscosity at baseline conditions (Measurement A, prior to incubation) was measured for each mucus sample (750 μl) Subsequently, each mucus sample was incubated at 37° C. for 30 minutes respectively with saline solution (50 μl) (Saline, used as control) and NAC (NAC in saline solution) coming from one of the following preparations: Composition 1 (70 μg) (NAC 71), Composition 2 (70 μg) (NAC71-FLM), Composition 3 (70 μg) (NAC75-FL), Composition 4 (70 μg) (NAC80-FL); thus, the viscosity measurement was carried out on each sample (Measurement B, after incubation) (table 2 and FIG. 1). Said viscosities (Measurement A and B) were measured at 37° C. and they are indicated in FIG. 1 as average viscosity value (ETA) in mPa·s.

The instrument used for measuring viscosity is a concentric cylinder rotational rheometer which applies the sinusoidal oscillations method (Mucorheometer ESLAB, Milan), which allows to obtain measurements on the mucus ranging between 500 mPa·s up to 80.000 mPa·s.

TABLE 1

| | Composition 1 | Composition 2 | Composition 3 | Composition 4 |
|---|---|---|---|---|
| NAC | 71% | 71% | 75% | 80% |
| L | / | 25% | 25% | 20% |
| M | / | 4% | / | / |

NAC N-acetylcysteine; L lactose; M magnesium stearate.
% by weight with respect to the total weight of the mixture M

Results

Table 2 shows the descriptive analysis of the viscosity measurements (in mPa·s. at 37° C.) before and after incubation (Measurements A and B) and represented in FIG. 1—with a histogram chart—are the average viscosity values (ETA, average viscosity values at 37° C.) indicated in table 2.

Table 3 shows the statistical analysis of the differences; the variance analysis was conducted using the Kruskal-Wallis test for repeated measurements and using the Dunnet test for post-hoc analysis (significant values, P<0.05): the differences were significant between pre- and post-treatment, while they are not significant between the various compositions of post-treatment of N-acetylcysteine (Composition 1-4).

TABLE 2

| | Saline | Seline | NAC 71 | NAC 71 | NAC 71 + | NAC 71 + | NAC 75 + | NAC 75 + | NAC 80 + | NAC 80 + |
|---|---|---|---|---|---|---|---|---|---|---|
| | B | A | B | A | LM B | LM A | L B | L A | L B | L A |
| Value number | 10 | 10 | 10 | 10 | 10 | 10 | 10 | 10 | 10 | 10 |
| Minimum | 4746 | 5530 | 4712 | 1905 | 4712 | 1905 | 4696 | 2136 | 4598 | 1789 |
| Percentile of 25% | 5634 | 6246 | 5756 | 2144 | 5756 | 2144 | 5965 | 2613 | 6487 | 1905 |
| Median | 5969 | 7231 | 7434 | 2395 | 7838 | 2709 | 7103 | 3037 | 7558 | 2163 |
| Percentile at 75% | 6724 | 8647 | 8806 | 3076 | 9107 | 3621 | 8796 | 4375 | 8356 | 2733 |
| Maximum | 9810 | 9826 | 9680 | 3232 | 9680 | 4136 | 9387 | 5322 | 8562 | 4506 |
| Average | 6279 | 7402 | 7354 | 2555 | 7477 | 2833 | 7158 | 3342 | 7217 | 2428 |
| Standard Deviation | 1406 | 1401 | 1700 | 501.2 | 1831 | 799.7 | 1529 | 1052 | 1237 | 823.5 |
| Standard error | 444.6 | 443.1 | 537.7 | 158.5 | 578.9 | 252.9 | 483.4 | 332.7 | 391 | 260.4 |
| Below 95% CI | 5273 | 6399 | 6138 | 2197 | 6167 | 2261 | 6065 | 2590 | 6332 | 1839 |
| of the average | | | | | | | | | | |
| Above 95% CI | 7284 | 8404 | 8571 | 2914 | 8786 | 3405 | 8252 | 4095 | 8101 | 3018 |
| of the average | | | | | | | | | | |
| Total | 62788 | 74015 | 73544 | 25554 | 74767 | 28328 | 71582 | 33424 | 72167 | 24285 |

TABLE 3

| | | Significance of |
|---|---|---|
| | | the median |
| Multiple comparative Dunn test | Difference | variation (P > 0.05) |
| Saline A vs NAC71 A | 55.40 | ** |
| Saline A vs NAC71 A + LM | 52.90 | ** |
| Saline A vs NAC75 A + L | 46.80 | * |
| Saline A vs NAC80 A + L | 60.10 | *** |
| NAC71 B vs NAC71 A | 53.35 | ** |
| NAC71 B + LM vs NAC71 A + LM | 52.45 | ** |
| NAC75 B + L vs NAC75 A + L | 42.30 | not significant |
| NAC80 B + L vs NAC80 A + L | 57.2 | *** |

*significant; ** more significant; *** even more significant.

Conclusions

The obtained results (table 2, table 3 and FIG. 1) show that the Compositions 1-4 according to the invention comprising N-acetylcysteine alone or combined with lactose and/or magnesium stearate in powder form for inhalation by means of oral aspiration are effective at reducing the viscosity of the bronchial mucus coming from patients suffering from cystic fibrosis.

Thus, the compositions of the invention are greatly effective in the treatment of bronchial mucus hypersecretion in the various diseases of the respiratory system.

Experimental Part (II)

The viscosity of the mucus coming from patients according to the conditions and methods described in the Experimental Part (I), was measured with the aim of the in vitro evaluation of the compositions according to the present invention (Composition 5-12 having the compositions indicated in table 4).

TABLE 4
NAC N-acetylcysteine; HA: hyaluronic acid; L lactose; M magnesium stearate;
% by weight with respect to the total weight of the mixture M.

| | Composition | | | | | | | | | | |
|---|---|---|---|---|---|---|---|---|---|---|---|
| | 5 | 6 | 7 | 8 | 9 | 10 | 11 | 12 | 13 | 14 | 15 |
| NAC | 5 | 10 | 20 | 5 | 10 | 20 | / | 80 | 5 | 10 | 20 |
| HA | 75 | 70 | 60 | 91 | 86 | 76 | 80 | / | / | / | / |
| L | 20 | 20 | 20 | / | / | / | 20 | 20 | / | / | / |
| M | / | / | / | 4 | 4 | 4 | / | / | / | / | / |

Results

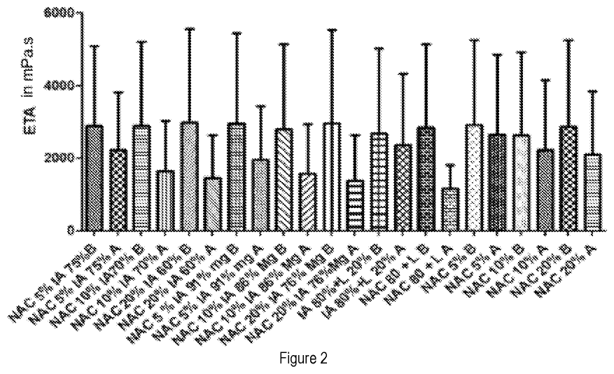

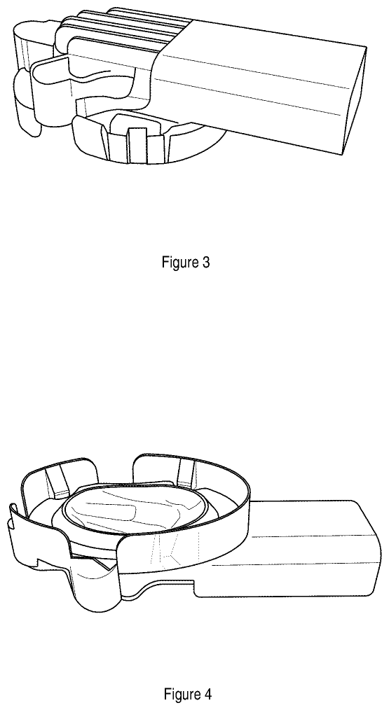

Table 5a and table 5b show the descriptive analysis of the viscosity measurements (in mPa·s. at 37° C.) before and after incubation (Measurements A and B) of Compositions 5-12 of table 4, and represented in FIG. 2—with a histogram chart—are the average viscosity values (ETA, average viscosity values at 37° C.) of Compositions 5-15, of table 4, indicated in table 6 for Compositions 5-12.

Table 6 shows the statistical analysis of the differences for Compositions 5-12; the variance analysis was conducted using the Kruskal-Wallis test for repeated measurements and using the Dunnet test for post-hoc analysis (significant values, P<0.05): the differences are significant from a statistical standpoint between pre- and post-treatment for a 10% and 20% concentration of NAC used. (FIG. 2)

TABLE 5a

| | | | NAC 5% | NAC 5% | NAC 10% | NAC 10% | NAC 20% | NAC 20% |
|---|---|---|---|---|---|---|---|---|
| | Saline B | Saline A | IA 75% B | IA 75% A | IA 70% B | IA 70% A | IA 60% B | IA 60% A |
| Value number | 11 | 11 | 11 | 11 | 11 | 11 | 11 | 11 |
| Minimum | 5979 | 6321 | 5765 | 3798 | 5895 | 3654 | 6854 | 2965 |
| Perrentile of 25% | 6785 | 6525 | 6528 | 4570 | 6846 | 3957 | 7598 | 3597 |
| Median | 7524 | 7674 | 6854 | 5428 | 7469 | 4258 | 7965 | 3827 |
| Percentile at 75% | 7871 | 8423 | 8623 | 6354 | 8524 | 4625 | 8733 | 4232 |
| Maximum | 8962 | 8854 | 8757 | 6842 | 8759 | 5321 | 8956 | 4429 |
| Average | 7457 | 7839 | 7279 | 5381 | 7480 | 4382 | 8106 | 3782 |
| Standard Deviation | 1867.9 | 914.6 | 1089 | 988.5 | 934.7 | 468.3 | 663.1 | 463.5 |
| Standard error | 261.7 | 275.8 | 328.4 | 297.4 | 281.8 | 141.2 | 199.9 | 139.8 |
| Below 95% CI | 6874 | 7024 | 6547 | 4728 | 6852 | 4067 | 7661 | 3471 |
| of the average | | | | | | | | |
| Above 95% CI | 8040 | 8253 | 8010 | 5054 | 8108 | 4696 | 8552 | 4093 |
| of the average | | | | | | | | |
| Total | 82025 | 84024 | 80064 | 59301 | 82279 | 48201 | 89188 | 41601 |

TABLE 5b

| | NAC 5% IA | NAC 5% IA | NAC 10% IA | NAC 10% IA | NAC 20% IA | NAC 20% IA | IA 80% + | IA 80% + | NAC | NAC |
|---|---|---|---|---|---|---|---|---|---|---|
| | 91% mg B | 91% mg A | 86% Mg B | 86% Mg A | 76% Mg B | 76% Mg A | L 20% B | L 20% A | 80 + L B | 80 + L A |
| Value number | 11 | 11 | 11 | 11 | 11 | 11 | 11 | 11 | 10 | 10 |
| Minimum | 6854 | 3830 | 6459 | 3735 | 8846 | 3584 | 5751 | 5231 | 4598 | 1789 |
| Percentile | 7420 | 4158 | 6859 | 3856 | 7585 | 3898 | 6928 | 6129 | 6487 | 1905 |
| of 25% | | | | | | | | | | |
| Median | 7866 | 4836 | 7430 | 4258 | 7952 | 3785 | 7523 | 6258 | 7558 | 2153 |
| Percentile | 8738 | 5689 | 7879 | 4558 | 5639 | 4124 | 8426 | 6586 | 6356 | 2733 |
| at 75% | | | | | | | | | | |
| Maximum | 8938 | 5828 | 8542 | 4957 | 8957 | 4258 | 8428 | 7265 | 6562 | 4506 |
| Average | 7912 | 4883 | 7483 | 4271 | 8087 | 3388 | 7359 | 6258 | 7217 | 2428 |
| Standard | 7349 | 739.9 | 695.1 | 379.5 | 633.2 | 237.2 | 531.1 | 613.4 | 1237 | 823.5 |
| Deviation | | | | | | | | | | |
| Standarderror | 2216 | 223.1 | 209.5 | 114.4 | 190.9 | 71.53 | 150.1 | 184.9 | 391 | 260.4 |
| Below 95% CI | 7419 | 4386 | 7018 | 4016 | 7671 | 3730 | 7012 | 5845 | 6332 | 1839 |
| of the average | | | | | | | | | | |
| Above 95% CI | 8408 | 5380 | 7950 | 4525 | 8522 | 4049 | 7723 | 6671 | 8081 | 3018 |
| of the average | | | | | | | | | | |
| Total | 87036 | 53718 | 82314 | 45976 | 89062 | 42784 | 81061 | 68843 | 72167 | 24285 |

TABLE 6

| | | Significance of |
|---|---|---|
| | | the median |
| Multiple comparative Dunn test | Difference | variation (P > 0.05) |
| Saline B vs Saline A | −6.364 | ns |
| Saline B vs NAC 5% IA 75% B | 6.636 | ns |
| Saline B vs NAC 5% IA 75% A | 66.18 | ns |
| Saline B vs NAC 10% IA70% B | −2.136 | ns |
| Saline B vs NAC 10% IA 70% A | 87.82 | * |
| Saline B vs NAC 20% IA 60% B | −28.09 | ns |
| Saline B vs NAC 20% IA 60% A | 107.5 | ** |
| Saline B vs NAC 5% IA 91% mg B | −18.45 | ns |
| Saline B vs NAC 5% IA 91% mg A | 77.18 | ns |
| Saline B vs NAC 10% IA 86% Mg B | −1.273 | ns |
| Saline B vs NAC 10% IA 86% Mg A | 91.09 | * |
| Saline B vs NAC 20% IA 76% Mg B | −26.68 | ns |
| Saline B vs NAC 20% IA 76% Mg A | 107.8 | ** |
| Saline B vs IA 80% + L 20% B | 6.182 | ns |
| Saline B vs IA 80% + L 20% A | 45.14 | ns |
| Saline B vs NAC 80 + L B | 5.959 | ns |
| Saline B vs NAC 80 + L A | 124.2 | *** |
| Saline A vs NAC 5% IA 75% B | 13.00 | ns |
| Saline A vs NAC 5% IA 75% A | 72.55 | ns |
| Saline A vs NAC 10% IA70% B | 4.227 | ns |
| Saline A vs NAC 10% IA 70% A | 94.18 | * |
| Saline A vs NAC 20% IA 60% B | −21.73 | ns |
| Saline A vs NAC 20% IA 60% A | 113.9 | *** |
| Saline A vs NAC 5% IA 91% Mg B | −12.09 | ns |
| Saline A vs NAC 5% IA 91% Mg A | 83.55 | ns |
| Saline A vs NAC 10% IA 86% Mg B | 5.091 | ns |
| Saline A vs NAC 10% IA 86% Mg A | 97.45 | ** |
| Saline A vs NAC 20% IA 76% Mg B | −20.32 | ns |
| Saline A vs NAC 20% IA 76% Mg A | 114.2 | *** |
| Saline A vs IA 80% + L 20% B | 12.55 | ns |
| Saline A vs IA 80% + L 20% A | 51.50 | ns |
| Saline A vs NAC 80 + L B | 12.32 | ns |
| Saline A vs NAC 80 + LA | 130.6 | *** |
| NAC 5% IA 75% B vs NAC 5% IA 75% A | 59.55 | ns |
| NAC 5% IA 75% B vs NAC 10% IA70% B | −8.773 | ns |
| NAC 5% IA 75% B vs NAC 10% IA 70% A | 81.18 | ns |
| NAC 5% IA 75% B vs NAC 20% IA 60% B | −34.73 | ns |
| NAC 5% IA 75% B vs NAC 20% IA 60% A | 100.9 | ** |
| NAC 5% IA 75% B vs NAC 5% IA 91% Mg B | −25.09 | ns |
| NAC 5% IA 75% B vs NAC 5% IA 91% Mg A | 70.55 | ns |
| NAC 5% IA 75% B vs NAC 10% IA 86% Mg B | −7.909 | ns |
| NAC 5% IA 75% B vs NAC 10% IA 86% Mg A | 84.45 | ns |
| NAC 5% IA 75% B vs NAC 20% IA 76% Mg B | −33.32 | ns |
| NAC 5% IA 75% B vs NAC 20% IA 76% Mg A | 101.2 | ** |
| NAC 5% IA 75% B vs IA 80% + L 20% B | −0.4545 | ns |
| NAC 5% IA 75% B vs IA 80% + L 20% A | 38.50 | ns |
| NAC 5% IA 75% B vs NAC 80% + L20% B | −0.6773 | ns |
| NAC 5% IA 75% B vs NAC 80% + L20% A | 117.6 | *** |
| NAC 5% IA 75% A vs NAC 10% IA70% B | −68.32 | ns |
| NAC 5% IA 75% A vs NAC 10% IA 70% A | 21.64 | ns |
| NAC 5% IA 75% A vs NAC 20% IA 60% B | −94.27 | * |
| NAC 5% IA 75% A vs NAC 20% IA 60% A | 41.36 | ns |
| NAC 5% IA 75% A vs NAC 5% IA 91% mg B | −84.64 | ns |
| NAC 5% IA 75% A vs NAC 5% IA 91% mg A | 11.00 | ns |
| NAC 5% IA 75% A vs NAC 10% IA 86% Mg B | −67.45 | ns |
| NAC 5% IA 75% A vs NAC 10% IA 86% Mg A | 24.91 | ns |
| NAC 5% IA 75% A vs NAC 20% IA 76% Mg B | −92.86 | * |
| NAC 5% IA 75% A vs NAC 20% IA 76% Mg A | 41.64 | ns |
| NAC 5% IA 75% A vs IA 80% + L 20% B | −60.00 | ns |
| NAC 5% IA 75% A vs IA 80% + L 20% A | −21.05 | ns |
| NAC 5% IA 75% A vs NAC 80% + L20% B | −60.22 | ns |
| NAC 5% IA 75% A vs NAC 80% + L20% A | 58.03 | ns |
| NAC 10% IA70% B vs NAC 10% IA 70% A | 89.95 | * |
| NAC 10% IA70% B vs NAC 20% IA 60% B | −25.95 | ns |
| NAC 10% IA70% B vs NAC 20% IA 60% A | 109.7 | *** |
| NAC 10% IA70% B vs NAC 5% IA 91% mg B | −16.32 | ns |
| NAC 10% IA70% B vs NAC 5% IA 91% mg A | 79.32 | ns |
| NAC 10% IA70% B vs NAC 10% IA 86% Mg B | 0.8636 | ns |
| NAC 10% IA70% B vs NAC 10% IA 86% Mg A | 93.23 | * |
| NAC 10% IA70% B vs NAC 20% IA 76% Mg B | −24.55 | ns |
| NAC 10% IA70% B vs NAC 20% IA 76% Mg A | 110.0 | *** |
| NAC 10% IA70% B vs IA 80% + L 20% B | 8.318 | ns |
| NAC 10% IA70% B vs IA 80% + L 20% A | 47.27 | ns |
| NAC 10% IA70% B vs NAC 80% + L20% B | 8.095 | ns |
| NAC 10% IA70% B vs NAC 80% + L20% A | 126.3 | *** |
| NAC 10% IA 70% A vs NAC 20% IA 60% B | −115.9 | *** |
| NAC 10% IA 70% A vs NAC 20% IA 60% A | 19.73 | ns |
| NAC 10% IA 70% A vs NAC 5% IA 91% mg B | −106.3 | ** |
| NAC 10% IA 70% A vs NAC 5% IA 91% mg A | −10.64 | ns |
| NAC 10% IA 70% A vs NAC 10% IA 86% Mg B | −89.09 | * |
| NAC 10% IA 70% A vs NAC 10% IA 86% Mg A | 3.273 | ns |
| NAC 10% IA 70% A vs NAC 20% IA 76% Mg B | −114.5 | *** |
| NAC 10% IA 70% A vs NAC 20% IA 76% Mg A | 20.00 | ns |
| NAC 10% IA 70% A vs IA 80% + L 20% B | −81.64 | ns |
| NAC 10% IA 70% A vs IA 80% + L 20% A | −42.68 | ns |
| NAC 10% IA 70% A vs NAC 80% + L20% B | −81.86 | ns |
| NAC 10% IA 70% A vs NAC 80% + L 20% A | 36.39 | ns |
| NAC 20% IA 60% B vs NAC 20% IA 60% A | 135.6 | *** |
| NAC 20% IA 60% B vs NAC 5% IA 91% mg B | 9.636 | ns |
| NAC 20% IA 60% B vs NAC 5% IA 91% mg A | 105.3 | ** |
| NAC 20% IA 60% B vs NAC 10% IA 86% Mg B | 26.82 | ns |
| NAC 20% IA 60% B vs NAC 10% IA 86% Mg A | 119.2 | *** |
| NAC 20% IA 60% B vs NAC 20% IA 76% Mg B | 1.409 | ns |
| NAC 20% IA 60% B vs NAC 20% IA 76% Mg A | 135.9 | *** |
| NAC 20% IA 60% B vs IA 80% + L 20% B | 34.27 | ns |
| NAC 20% IA 60% B vs IA 80% + L 20% A | 73.23 | ns |
| NAC 20% IA 60% B vs NAC 80% + L20% B | 34.05 | ns |
| NAC 20% IA 60% B vs NAC 80% + L20% A | 152.3 | *** |
| NAC 20% IA 60% A vs NAC 5% IA 91% mg B | −126.0 | *** |
| NAC 20% IA 60% A vs NAC 5% IA 91% mg A | −30.36 | ns |
| NAC 20% IA 60% A vs NAC 10% IA 86% Mg B | −108.8 | ** |
| NAC 20% IA 60% A vs NAC 10% IA 86% Mg A | −16.45 | ns |
| NAC 20% IA 60% A vs NAC 20% IA 76% Mg B | −134.2 | *** |
| NAC 20% IA 60% A vs NAC 20% IA 76% Mg A | 0.2727 | ns |
| NAC 20% IA 60% A vs IA 80% + L 20% B | −101.4 | ** |
| NAC 20% IA 60% A vs IA 80% + L 20% A | −62.41 | ns |
| NAC 20% IA 60% A vs NAC 80% + L20% B | −101.6 | ** |
| NAC 20% IA 60% A vs NAC 80% + L20% A | 16.66 | ns |
| NAC 5% IA 91% mg B vs NAC 5% IA 91% mg A | 95.64 | * |
| NAC 5% IA 91% mg B vs NAC 10% IA 86% Mg B | 17.18 | ns |
| NAC 5% IA 91% mg B vs NAC 10% IA 86% Mg A | 109.5 | *** |
| NAC 5% IA 91% mg B vs NAC 20% IA 76% Mg B | −8.227 | ns |
| NAC 5% IA 91% mg B vs NAC 20% IA 76% Mg A | 126.3 | *** |
| NAC 5% IA 91% mg B vs IA 80% + L 20% B | 24.64 | ns |
| NAC 5% IA 91% mg B vs IA 80% + L 20% A | 63.59 | ns |
| NAC 5% IA 91% mg B vs NAC 80% + L20% B | 24.41 | ns |
| NAC 5% IA 91% mg B vs NAC 80% + L20% A | 142.7 | *** |
| NAC 5% IA 91% mg A vs NAC 10% IA 86% Mg B | −78.45 | ns |
| NAC 5% IA 91% mg A vs NAC 10% IA 86% Mg A | 13.91 | ns |
| NAC 5% IA 91% mg A vs NAC 20% IA 76% Mg B | −103.9 | ** |
| NAC 5% IA 91% mg A vs NAC 20% IA 76% Mg A | 30.64 | ns |
| NAC 5% IA 91% mg A vs IA 80% + L 20% B | −71.00 | ns |
| NAC 5% IA 91% mg A vs IA 80% + L 20% A | −32.05 | ns |
| NAC 5% IA 91% mg A vs NAC 80% + L20% B | −71.22 | ns |
| NAC 5% IA 91% mg A vs NAC 80% + L20% A | 47.03 | ns |
| NAC 10% IA 86% Mg B vs NAC 10% IA 86% Mg | 92.36 | * |
| A | | |
| NAC 10% IA 86% Mg B vs NAC 20% IA 76% Mg | −25.41 | ns |
| B | | |
| NAC 10% IA 86% Mg B vs NAC 20% IA 76% Mg | 109.1 | *** |
| A | | |
| NAC 10% IA 86% Mg B vs IA 80% + L 20% B | 7.455 | ns |
| NAC 10% IA 86% Mg B vs IA 80% + L 20% A | 46.41 | ns |
| NAC 10% IA 86% Mg B vs NAC 80% + L 20% B | 7.232 | ns |
| NAC 10% IA 86% Mg B vs NAC 80% + L20% A | 125.5 | *** |
| NAC 10% IA 86% Mg A vs NAC 20% IA 76% Mg | −117.8 | *** |
| B | | |
| NAC 10% IA 86% Mg A vs NAC 20% IA 76% Mg | 16.73 | ns |
| A | | |
| NAC 10% IA 86% Mg A vs IA 80% + L 20% B | −84.91 | ns |
| NAC 10% IA 86% Mg A vs IA 80% + L 20% A | −45.95 | ns |
| NAC 10% IA 86% Mg A vs NAC 80% + L20% B | −85.13 | ns |
| NAC 10% IA 86% Mg A vs NAC 80% + L20% A | 33.12 | ns |
| NAC 20% IA 76% Mg B vs NAC 20% IA 76% Mg | 134.5 | *** |
| A | | |
| NAC 20% IA 76% Mg B vs IA 80% + L 20% B | 32.86 | ns |
| NAC 20% IA 76% Mg B vs IA 80% + L 20% A | 71.82 | ns |
| NAC 20% IA 76% Mg B vs NAC 80% + L20% B | 32.64 | ns |
| NAC 20% IA 76% Mg B vs NAC 80% + L 20% A | 150.9 | *** |
| NAC 20% IA 76% Mg A vs IA 80% + L 20% B | −101.6 | ** |
| NAC 20% IA 76% Mg A vs IA 80% + L 20% A | −62.68 | ns |
| NAC 20% IA 76% Mg A vs NAC 80% + L20% B | −101.9 | ** |
| NAC 20% IA 76% Mg A vs NAC 80% + L20% A | 16.39 | ns |
| IA 80% + L 20% B vs IA 80% + L 20% A | 38.95 | ns |
| IA 80% + L 20% B vs NAC 80% + L20% B | −0.2227 | ns |
| IA 80% + L 20% B vs NAC 80% + L20% A | 118.0 | *** |
| IA 80% + L 20% A vs NAC 80% + L20% B | −39.18 | ns |
| IA 80% + L 20% A vs NAC 80% + L20% A | 79.07 | ns |
| NAC 80% + L20% B vs NAC 80% + L20% A | 118.3 | *** |

ns: not significant, *significant; ** more significant; ***even more significant.

Conclusions

The obtained results show that both acetylcysteine (NAC) alone and in association with hyaluronic acid (HA) revealed to be effective at reducing the viscosity of bronchial mucus. NAC breaks the disulphide bonds of the glycoprotein agglomerates by chemically breaking the glycoprotein structures into less viscous components.

At low concentrations of NAC there are no significant effects, for example Composition 5 comprising 5% by weight of NAC. However, the addition of HA was showed increase the effect of NAC, hence compositions comprising NAC at 10% or 20% are effective.

Thus, a medical device that provides NAC and HA in form of dry powder for inhalation by means of oral aspiration can be very useful in the treatment of bronchial hypersecretion in various respiratory diseases.

1. A method for preventive and/or curative treatment of mucus hypersecretion and of a disease, symptom and/or disorder associated with said mucus hypersecretion, comprising:
administering to a subject in need thereof a composition in a dry powder form comprising:
(i) a mixture M comprising, or alternatively, consisting of a mucolytic agent and, optionally,
(ii) at least one acceptable pharmaceutical or food grade additive and/or excipient, wherein said mucolytic agent is N-acetylcysteine or an acceptable pharmaceutical or food grade salt thereof and wherein said composition is administered through oral inhalation activated by an aspiration action by the subject.

2. The method according to claim 1, wherein said (i) mixture M further comprises a hyaluronic acid or an acceptable pharmaceutical or food grade salt thereof.

3. The method according to claim 1, wherein said (i) mixture M further comprises a first support agent selected from among the group comprising or, alternatively, consisting of lactose, a dextran, mannitol and the mixtures thereof; preferably lactose.

4. The method for use according to claim 1, wherein said (i) mixture M further comprises a second support agent, wherein said second agent is a stearate metal or a derivative thereof selected from among the group comprising or, alternatively, consisting of: magnesium stearate, zinc stearate, calcium stearate, sodium stearate, lithium stearate, sodium stearyl fumarate, sodium stearoyl lactylate and the mixtures thereof; preferably magnesium stearate.

5. The method for use according to claim 1, wherein the (i) mixture M comprises or, alternatively, consists of N-acetylcysteine and hyaluronic acid, or the salts thereof, and, furthermore, lactose and/or magnesium stearate.

6. The method for use according to claim 1, wherein the (i) mixture M comprises or, alternatively, consists of a % by weight of N-acetylcysteine, or a salt thereof, comprised in the range between 0.1% and 60% with respect to the total weight of the mixture M, preferably between 5% and 50%, more preferably between 10% and 40%.

7. The method according to claim 2, wherein said (i) mixture M comprising, or alternatively, consisting of % by weight of N-acetylcysteine, or a salt thereof, comprised in the range between 0.1% and 60% with respect to the total weight of the mixture M, preferably between 5% and 50%, more preferably between 10% and 40%; and a % by weight of hyaluronic acid, or a salt thereof, comprised in the range between 40% and 90% with respect to the total weight of the mixture M, preferably between 50% and 80%, more preferably between 60% and 80%.

8. The method according to claim 3, wherein the (i) mixture M comprises or, alternatively, consists of:
a % by weight of N-acetylcysteine, or a salt thereof, comprised in the range between 0.1% and 58% with respect to the total weight of the mixture M, preferably between 5% and 50%, more preferably between 10% and 40%; and a % by weight of hyaluronic acid, or a salt thereof, comprised in the range between 40% and 90% with respect to the total weight of the mixture M, preferably between 50% and 80%, more preferably between 60% and 80%; and a % by weight of a first support agent, preferably lactose, and/or of a second support agent, preferably magnesium stearate, comprised in the range between 2% and 35% with respect to the total weight of the mixture M, preferably between 10% and 30%, more preferably between 15% and 25%.

9. The method according to claim 1, wherein said disease, symptom and/or disorder associated with said mucus hypersecretion is an inflammation or infection or allergy of the respiratory system.

10. The method according to claim 9, wherein said inflammation or infection or allergy of the respiratory system is selected from among the group comprising or, alternatively, consisting of: asthma, chronic obstructive pulmonary disease (COPD), bronchitis, emphysema, cystic fibrosis, pertussis, pneumonia, pleuritis, bronchiolitis, cold, sinusitis, rhinitis, tracheitis, pharyngitis, laryngitis, epiglottitis and bronchiectasis.

11. A dispensing device for the administration of a dry powder through the oral-inhalation route, wherein said device comprises
a compartment for containing the powder comprising the composition in form of dry powder according to claim 1,
an aspiration channel, wherein a first end is directly or indirectly connected to the powder containment compartment and a second end which allows the subject to carry out an oral aspiration action;
wherein said powder containment compartment is loaded with said composition in form of dry powder by means of a loader comprising said composition, and wherein the device is structured so as to dispense a dose of said composition following said oral aspiration action by the subject in need of said administration.

12. The device according to claim 11, wherein said loader is of the single-dose or multi-dose type.

13. The device according to claim 11, wherein the device further comprises a containment compartment of a loader positioned in which is said loader comprising said composition in form of dry powder.

14. The device according to claim 11, wherein said loader is an external loader positioned outside the device, and wherein the device is structured so as to allow the direct transfer of a single-dose of said composition from the external loader to the powder containment compartment.

15. The device according to claim 13, wherein said loader is an internal loader positioned inside the device in said containment compartment of the loader, and wherein the device is structured so as to allow the indirect transfer of a single-dose of said composition from the external loader to the powder containment compartment actuating a pressure on the device or on the internal loader which causes the opening of the internal loader.

16. An inhalable dry powder comprising:
(i) a mixture M comprising, or alternatively, consisting of a mucolytic agent and, optionally,
(ii) at least one acceptable pharmaceutical or food grade additive and/or excipient, wherein said mucolytic agent is N-acetylcysteine or an acceptable pharmaceutical or food grade salt thereof
